(19) United States
(12) Patent Application Publication
(10) Pub. No.: US 2016/0114109 A1
(43) Pub. Date: Apr. 28, 2016

(54) SYSTEM AND METHOD FOR PREPARING AND DELIVERING A MEDICAMENT

(71) Applicant: ROSS A.M.S. - ADVANCED MEDICATION SOLUTIONS LTD., Ramat-HaSharon (IL)

(72) Inventor: Gilad LAVI, Rishon-LeZion (IL)

(21) Appl. No.: 14/890,633

(22) PCT Filed: May 14, 2014
(86) PCT No.: PCT/IL14/50424

§ 371 Date: Nov. 12, 2015

Related U.S. Application Data
(60) Provisional application No. 61/825,134, filed on May 20, 2013.

(51) Int. Cl. A61M 5/315; A61M 5/20

(52) U.S. Cl. CPC A61M 5/31596; A61M 5/31515; A61M 5/20; A61M 2005/3152; A61M 2205/3592

(57) ABSTRACT

A system for medicament preparation and delivery is provided. The system includes a housing having a chamber for containing a liquid and a plunger movable within the chamber for drawing and dispensing liquid. The plunger and the housing are configured such that the plunger is movable via a drive mechanism capable of engaging a side of the plunger or alternatively by applying a force to a top of a shaft of the plunger.

### FIELD AND BACKGROUND OF THE INVENTION

The present invention relates to a system and method for preparing and delivering medicaments and, more particularly, to a syringe based system for compounding and administering drugs in health care settings.

Medication preparation and administration errors are the single most common preventable cause of adverse events in medication practice and a major public health burden, threatening the life of many patients. Medication errors may vary and can occur throughout the medication procedure: from prescribing the wrong drug, preparation mistake or incorrect administration of the medication.

Medical practice in recent years is characterized by an increase in patient safety awareness resulting in a vast surge in safety and technical measures. Hospitals and care givers are now implementing use of smart pumps, computerized medication software, automatic medication dispensing systems, pens injectors for home care settings, automatic pharmacy compounding robots and the like.

Despite these improvements, patient care still suffers from safety problems especially in the field of drug preparation and delivery. While some pharmacies have introduced expensive, complex, inflexible i.v. robotic preparation systems, the overwhelming majority of preparations are done manually relying on the abilities of technicians and nurses. Manual preparation and administration of medicaments is difficult, slow, labor intensive, undocumented, and prone to costly mistakes.

Medications in the form of liquid or powder are often supplied within rigid vials. A drug powder is reconstituted using a predetermined volume of a diluent withdrawn from a diluent vial or container. The diluent is then injected into the drug vial via a syringe, the drug vial is swirled, and the reconstituted medication is withdrawn back into the syringe which is then used to deliver the drug to the patient via the preferred method of administration. Due to the limitations of manual preparation, automated drug preparation systems such as Riva produced by Intelligent Hospital Systems or Health Robotics' i.v. Station find increasing use in hospitals. Such systems reduce overall medication errors providing a safer, more accurate way to prepare drugs, however, these systems are costly, require a large space in the pharmacy (often a dedicated room), can only handle a limited variety of drugs, and offer limited flexibility.

Thus, there is a need for a low cost, low impact drug compounding and administration system that follows traditional preparations techniques and can be used in hospital pharmacies and administration areas and can provide pharmacists, technicians, nurses and patients at home care, with a simple, fast, accurate, safe and documented approach for preparing and administering drugs.

### SUMMARY OF THE INVENTION

According to one aspect of the present invention there is provided a system for medicament preparation and delivery comprising: (a) a housing including a chamber for containing a liquid; (b) a plunger movable within the chamber for drawing and dispensing liquid, the plunger and the housing being configured such that the plunger is movable via a drive mechanism capable of engaging a side of the plunger or alternatively by applying a force to a top of a shaft of the plunger.

According to further features in preferred embodiments of the invention described below, the system further comprises a toggle for switching between movement of the plunger via the drive mechanism or movement of the plunger via the force to the top of the shaft of the plunger.

According to still further features in the described preferred embodiments the shaft of the plunger is configured with at least one groove for engaging the drive mechanism.

According to still further features in the described preferred embodiments the at least one groove is a spiral groove.

According to still further features in the described preferred embodiments the at least one groove engages a drive element of the drive mechanism.

According to still further features in the described preferred embodiments the toggle is capable of engaging the at least one groove.

According to still further features in the described preferred embodiments the system further comprises the drive mechanism.

According to still further features in the described preferred embodiments the drive mechanism is attachable to a side wall of the housing.

According to still further features in the described preferred embodiments the drive mechanism includes a motor having a drive gear.

According to still further features in the described preferred embodiments the drive gear is a pinion.

According to still further features in the described preferred embodiments the drive gear is a worm drive gear.

According to still further features in the described preferred embodiments the drive gear is capable of engaging at least one groove in the shaft of the plunger.

According to still further features in the described preferred embodiments the shaft releasably engages a shaft gear which is capable of engaging the drive gear.

According to still further features in the described preferred embodiments the top of the shaft is connectable to a manually operable plunger interface.

According to still further features in the described preferred embodiments the drive mechanism includes a control unit having a user interface for inputting parameters related to drawing and optionally dispensing of the liquid.

According to still further features in the described preferred embodiments the control unit includes an optical reader for scanning a drug vial.

According to still further features in the described preferred embodiments the control unit includes wireless communication capabilities or RFID.

According to still further features in the described preferred embodiments a proximal end of the shaft is configured capable of connecting to a spring driven mechanism.

According to still further features in the described preferred embodiments the system further comprises spring driven mechanism connectable to a proximal end of the shaft of the plunger and the housing, the spring driven mechanism is capable of applying the force to the top of the shaft of the plunger.

According to still further features in the described preferred embodiments the spring driven mechanism drives the shaft for delivering a liquid from the chamber.

According to still further features in the described preferred embodiments a tension of a spring of the spring driven mechanism is user adjustable.

According to still further features in the described preferred embodiments connection of the spring driven mechanism to the shaft and the housing prevents the drive mechanism from moving the plunger.

The present invention successfully addresses the shortcomings of the presently known configurations by providing a drug preparation and delivery system that can be used by health care providers and patients to enable safe, precise and effective compounding and delivery of drugs from a single unit.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1-2 illustrate exploded (FIG. 1) and assembled (FIG. 2) views of one embodiment of the present system.

FIG. 3 illustrates the present system with attached motorized drive mechanism.

FIGS. 4A-C illustrates isometric (FIG. 4A), side sectional (FIG. 4B) and top sectional (FIG. 4C) views of the present system with attached motorized drive mechanism and control unit.

FIG. 5 illustrates the shaft gear of the present system.

FIGS. 6A-C illustrate the present system with one embodiment of an attached manual plunger driver mechanism.

FIGS. 7A-C illustrate the present system with another embodiment of an attached manual plunger driver mechanism.

FIGS. 8A-D illustrate the present system with one embodiment of an attached spring loaded plunger driver mechanism.

### DESCRIPTION OF THE PREFERRED EMBODIMENTS

The present invention is of a system which can be used to prepare and dispense medication using a single chamber and two separate drive mechanisms.

The principles and operation of the present invention may be better understood with reference to the drawings and accompanying descriptions.

Before explaining at least one embodiment of the invention in detail, it is to be understood that the invention is not limited in its application to the details set forth in the following description. The invention is capable of other embodiments or of being practiced or carried out in various ways. Also, it is to be understood that the phraseology and terminology employed herein is for the purpose of description and should not be regarded as limiting.

Drug preparation and delivery systems are known in the art. Such systems typically include automated (robotic) free standing mixing control units which are capable of reconstituting and compounding drugs and loading medicament delivery devices such as syringes, pumps or i.v. bags.

Bench top or handheld syringe driver systems have also been described in the prior art (see, for example, U.S. Pat. No. 6,551,277; U.S. Pat. No. 6,428,509 and U.S. Pat. No. 5,236,416). Although such systems can be used to draw diluents and reconstitute drugs, they are typically utilized for controlling medicament delivery from an attached syringe, i.e. they primarily function as drug delivery pumps.

Such bench top systems typically utilize a driver assembly that connects directly to the finger hold of a standard syringe and as such are of limited accuracy and adaptability to various drug delivery mechanisms.

While reducing the present invention to practice, the present inventors have devised a drug dosing, reconstitution and delivery system that uses a single device chamber for drug preparation and delivery. The present system includes a single chamber fitted with a plunger and two distinct plunger-driving mechanisms, each utilizing a specific driver interface with the plunger. Such a system enables a user to semi-automatically reconstitute a medicament using a first plunger driving mechanism and deliver the medicament using a manual or spring loaded (second) plunger driving mechanism.

Thus, according to one aspect of the present invention there is provided a system for medicament preparation and delivery. As used herein, medicament preparation refers to reconstitution of a drug powder with a diluent and/or to compounding of liquid drugs.

The present system includes a housing having a chamber for containing a liquid and a plunger movable within the chamber for drawing and dispensing liquid.

The plunger and housing are configured such that the plunger is movable via a drive mechanism capable of engaging a side of the plunger. As is further described hereinunder, such a driver mechanism attaches to a side wall of the housing and mates with the side of the plunger shaft (which is optionally fitted with a shaft gear). The plunger is also configured for operation by applying a force to a top of a shaft of the plunger. As is further described hereinunder, such a force can be applied by a finger of the user or via a spring or motor driver plunger driver.

Such a dual-drive, single chamber configuration provides several advantages over prior art syringe drivers:

(i) driving plunger movement via a side-mating drive mechanism applies a force closer to the plunger head (that seals the chamber), thus minimizing forces that can displace (deflect) the plunger shaft from the movement axis;

(ii) radial support is provided by the plunger shaft itself, thus no external rails or drive guides are needed;

(iii) the drive mechanism is static, while the plunger moves past the drive mechanism;

(iv) a side-mating drive mechanism enables more accurate and fine control over plunger withdrawal without requiring complicated drive mechanisms;

(v) a side-mating drive mechanism does not engage the end of the plunger shaft which can then be designed and used for mating with plunger driving accessories, e.g. spring loaded drivers, hand operation drivers etc.; and

(vi) a side-mating drive mechanism substantially reduces the bulk and footprint of the system;

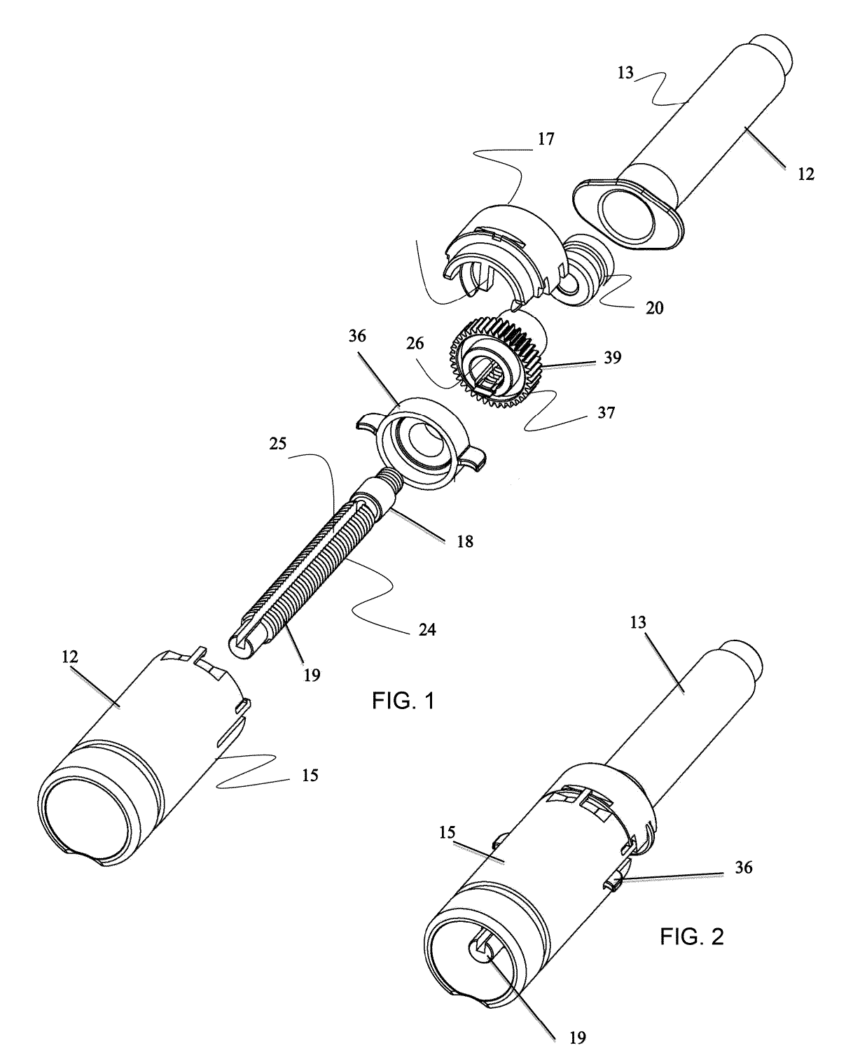

Embodiments of the present system, which is referred to herein as system 10, are illustrated in FIGS. 1-8c.

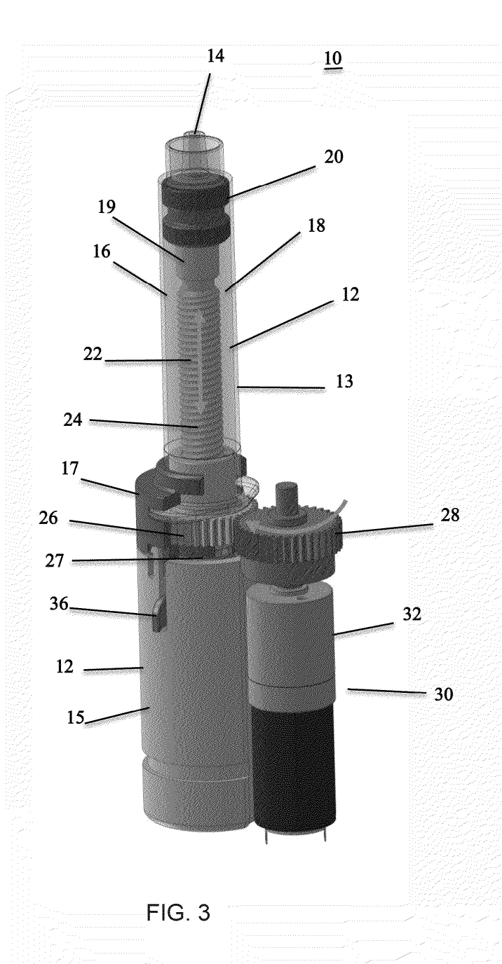

FIGS. 1-2 illustrate an embodiment of system 10 which includes a screw drive mechanism for moving a plunger within a barrel-shaped housing. Although such a mechanism is presently preferred, it will be appreciated that alternative mechanism including a worm drive, a ratchet drive, a friction drive and the like can also be used with the present invention.

FIG. 1 is an exploded view of system 10 showing the internal and external components. FIG. 2 is an assembled view of system 10.

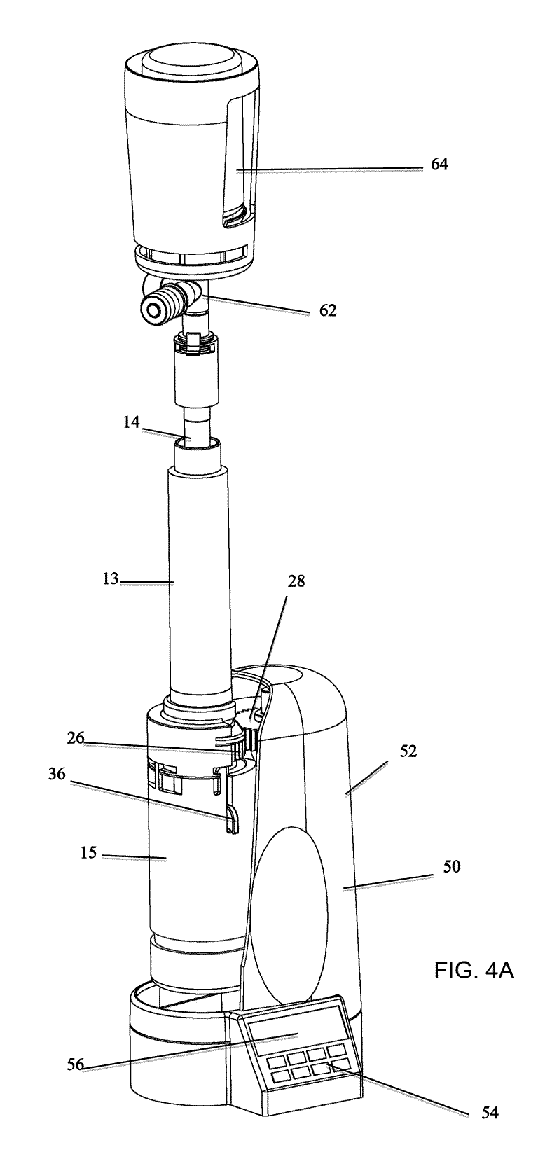

System 10 includes a housing 12 having a barrel-shaped configuration with port 14 (shown in FIG. 3) fitted at a distal end thereof for transferring fluid in and out of chamber 16. Port 14 can include a locking mechanism (e.g. Luer lock) for connecting a needle, vial adapter, tubing and the like.

In this embodiment of system 10, housing 12 includes a distal portion 13 that has a syringe like configuration and a proximal portion 15 which is barrel-shaped and larger in diameter than portion 13. Portions 13 and 15 of housing 12 can be co-formed as a single body, or preferably formed from two detachable parts (as shown in FIG. 1). In the latter case, portions 13 and 15 can be attached via flange 17.

Housing 12 is fabricated from a polymer such as polypropylene and is preferably transparent to enable viewing of the contents of chamber 16. Housing 12 can alternatively be fabricated from an alloy (e.g. stainless steel) in which case a transparent window is preferably configured along the length of housing 12.

A plunger 18 removably positioned within chamber 16 includes a plunger shaft 19 connected to a plunger head 20 which forms a seal with the internal walls of chamber 16. Head 20 is formed from an elastic material such as rubber (e.g. bromobutyl) or silicone and can include one or more contact interfaces with the walls of chamber 16 (two shown). Movement of plunger head 20 along a longitudinal axis of chamber 16 (as noted by double headed arrow of FIG. 3) defines the volume of chamber 16. Chamber 16 can have a volume of 1-60, 2-30, 3-25, 4-15 or 5-10 ml (e.g. 1 ml, 3 ml , 5 ml, 10 ml, 20 ml , 30 ml, 60 ml) when plunger head 20 is fully retracted (withdrawn). A preferred internal diameter of chamber can be selected from a range of 4-25, 5-20, 6-18, 10-15 mm. A preferred length of chamber 16 can be selected from a range of 60-150, 65-120, 80-100 mm.

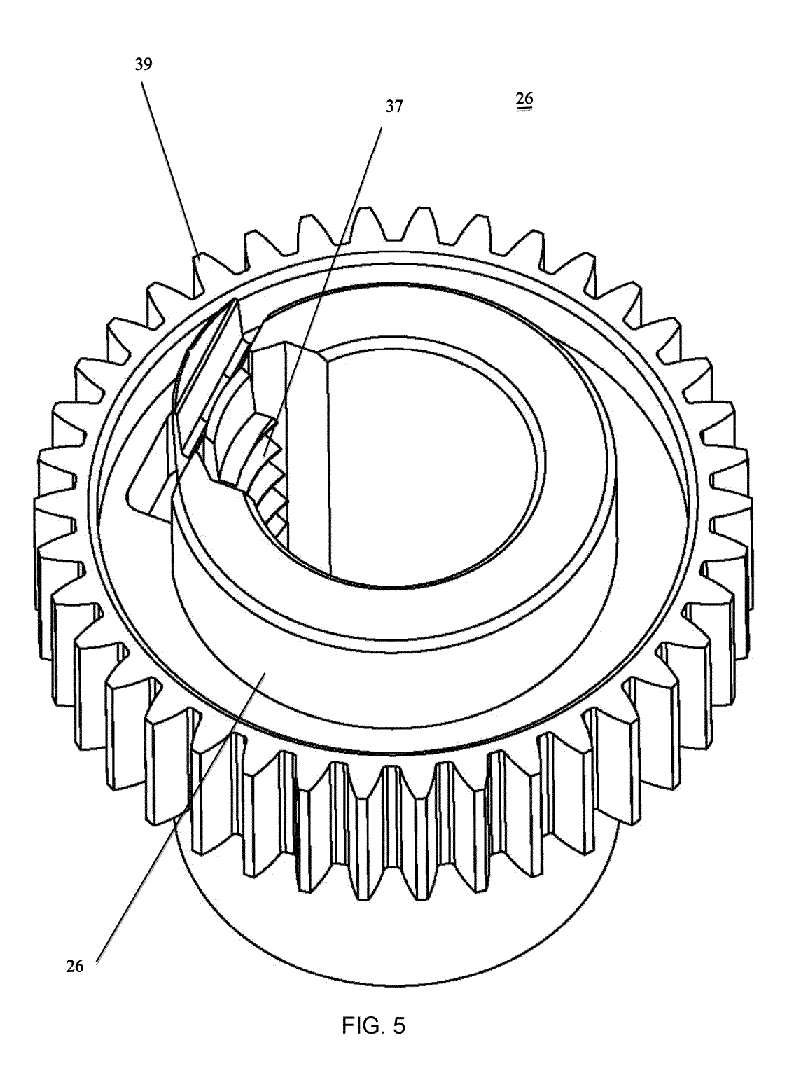

As is mentioned hereinabove, the present system is unique in that it employs a side mounted drive mechanism which mates with a side of the plunger shaft 19. To enable side driving, plunger shaft 22 is configured with at least spiral groove 24 (forming one or more drive coils) and notch 25 along its length, a shaft gear 26 is fitted over a proximal end portion of shaft 22. Shaft gear 26 includes internal teeth 37 (FIG. 5) and external drive teeth 39 (FIGS. 1 and 3). Internal teeth (thread) 37 of shaft gear 26 are arranged perpendicularly to external teeth 39 and are designed to mate with spiral groove 24 such that when shaft gear 26 is rotated around its axis, plunger head 20 slides within chamber 16 to increase or decrease the volume thereof. Internal teeth 37 can be configured with any spacing depending on the coil pitchy and number of coils formed by spiral groove 24. External teeth 39 can number between 15-50, preferably 20.

As is shown in FIG. 3, the external gear teeth of shaft gear 26 are exposed through a window 27 formed between portion 13 and 15 of housing 12. These gear teeth mate with a drive gear 28 of a removably attached drive mechanism 30. Drive mechanism 30 also includes an electric motor 32 connected to drive gear 28. Electric motor 32 can be a continuous or step motor (or encoder) such as a FaulHaber motor.

The drive ratio between drive gear 28 and shaft gear 26 can be anywhere between 1:1 to 5:1 (respectively), depending on the type of motor used and its internal drive (direct or geared). The spacing of spiral groove 24 and the dimensions (most notably the diameter) of the chamber. For example, a system 10 utilizing a motor that can turn at 10,000 rpm and is internally geared down by 20 to rotate at 500 rpm with a 1:1 transmission ration between drive gear 28 and shaft gear 26, and two spaced apart spiral grooves 24 with a pitch of 2.5 mm per turn, can drive plunger 18 at 20.8 mm/sec [500×2.5)/60]. Full travel of a standard 5 ml chamber syringe plunger is 40 mm and so complete withdrawal of plunger 18 can be effected in less than 2 seconds. The pitch of spiral groove 24 can be reduced in order to increase resolution. Such a motor can provide a maximal torque of 2.5 mNm, that enables it to provide approximate 50N of axial pulling or pushing force on plunger 18. In a 5 ml syringe chamber each ml is equal to 8 mm of axial travel (of plunger). Typical delivery accuracy is +/−5% or less. In the present system, the motor can be slowed down to increase accuracy (+/−0.1 mm of axial plunger travel).

Thus, system 10 can move plunger 18 in an accurate, fast manner considering maximal expected force during withdrawal and delivery. Since withdrawal rate and accuracy depends on gearing, spiral groove 24 pitch and chamber diameter, parameters that can be modified, system 10 provides the flexibility necessary to meet all the requirements of drug preparation and delivery.

In addition to the above, system 10 can include strain/load sensors (e.g. in plunger head 20 or between plunger head 20 and shaft 19) which can enable measurements of axial loads and determination of end of withdrawal of delivery or any potential malfunction (e.g. withdrawal forces higher than expected for a formulation based on formulation viscosity etc. may cause vacuum voids within the drug and increase the chances for air presence).

Alternatively, such sensing can be integrated into the drive mechanism to identify variations in strain on motor 32 (via current sensing), on gears 26 and/or 28 and the like.

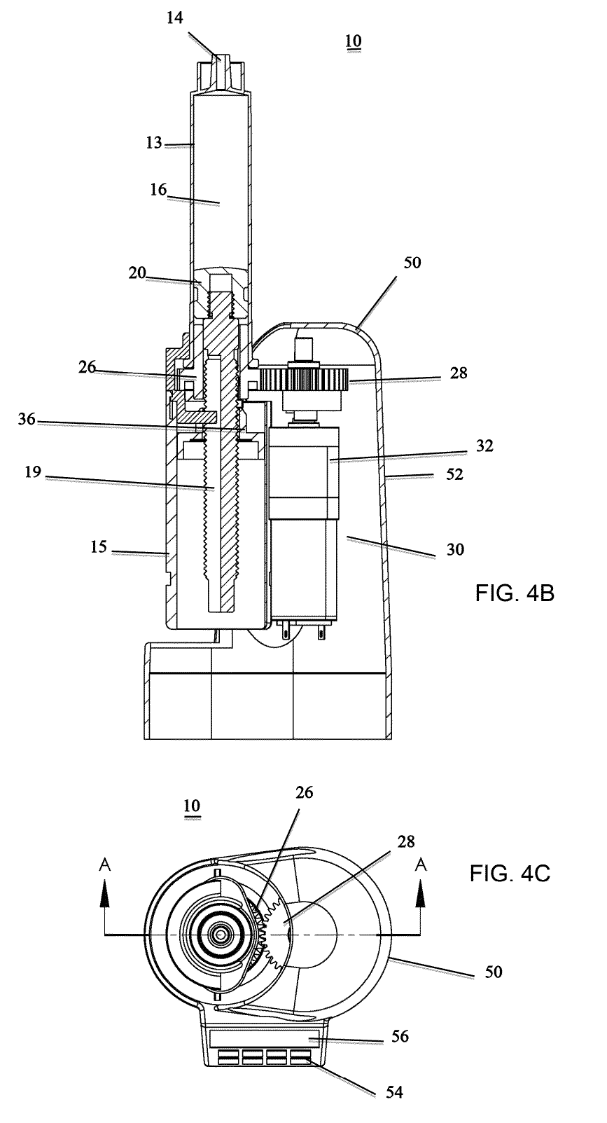

FIGS. 4a-c illustrate system 10 attached to a control unit 50. Control unit 50 includes a housing 52 for containing a microprocessor (executing a dedicated or open source operating system), wireless connectivity (e.g. Bluetooth, cellular, WiFi and the like), a rechargeable battery, gyroscope and accelerometer sensors, a proximity sensor, and ambient light sensor, data/power ports and the like.

A keypad 54 (push/touch controls) for entering information and a display 56 (e.g. LCD. LED, OLED etc), for providing a user with information are mounted in housing 52. Housing 52 can also incorporate a reader for imaging or scanning printed vial labels or for obtaining RFID information or by video imaging and a local UV light vial sterilizer unit.

Control unit 50 can provide a user with the following:

(i) two way communication with the hospital CPOE system or with a dedicated software;

(ii) closed loop communication with electronic prescription systems;

(iii) drug/diluent local or remote verification via image detection, bar code or RFID reading;

(iv) local/remote setting of medication ingredients and dosage;

(v) local/remote verification of medication ingredients/dosage;

(vi) electronically controlled dosing (control over withdrawal or injection of drug/diluent);

(vii) empirical/video imaging verification of drug reconstitution;

(viii) textual/audio alerts;

(ix) graphic/image guidance of preparation including vial drugs images and a full graphical guidance of the preparation stages;

(x) documentation of parental perpetrations;

(xi) syringe tagging by RFID or labeling of preparation details, patient ID administration route, administration time; and/or

(xii) administration verification and documentation.

(xiii) communication with a dedicated Smartphone application (of the patient or caregiver) for on-line medication authentication of the preparation and administration Process.

The following scenario describes one typical use of features (xi and xiii) described above. A drug prescription is received from the hospital's prescription system (CPOE) or from dedicated software. The prescription is verified and matched with the patient ID and profile by control unit 50. The correct dose is withdrawn and the syringe is associated (e.g. tagged with RFID) with data, such as, patient ID, administration route, administration rate and administration time [as is described in feature (xi) above]. The tagged syringe is loaded into system 10 which verifies that the medication is administered at the right time and rate. Once the drug is delivered, a message is sent to the Smartphone application.

As is shown in FIG. 4, system 10 can be used to reconstitute and draw medication from any type of vial. In order to prevent contamination and control medication reconstitution and compounding, port 14 is preferably fluidly connected to medication container 64 (e.g. vial) through adaptor 62 (FIG. 4). Examples of vial adaptors that can be used with the present invention include, but are not limited to, vial adaptors marketed by Westpharma, or Baxter's Inter link universal vial adapter, ICU medical's Multi Dose vial adapter and the like.

A vial adaptor 62 includes a spiking element having at-least one fluid channel and several brackets for securing the vial neck. Adapter 62 can also include a “skirt” like element for connecting the vial to additional components such as a reservoir and the like.

Since a vial can be used several times for drug withdrawal, vial adaptor 62 is preferably resealable (Luer seal) and can be wiped clean prior to engagement with a vial. Vial adaptor 62 is also configured for preventing leakage and for minimizing dead volume.

As described herein, system 10 also includes a second (separate) drive mechanism which is operated from the proximal end of plunger shaft 19. In order to enable use of this second drive mechanism, system 10 includes a locking switch 36 which locks shaft 19 to shaft gear 26 for operation via drive mechanism 30, and unlocks shaft 19 from shaft gear 26 for operation via the second drive mechanism. Locking switch 36 engages shaft 19 to shaft gear 26 when drive mechanism 30 and portion 15 of housing 12 are engaged with portion 13. When drive mechanism 30 and its attached portion 15 are removed (along with locking switch 36), shaft gear 26 (FIG. 5) disengages from notch 25 allowing shaft 19 to move freely in an out of shaft gear 26.

The second drive mechanism can be a manual, spring loaded or electrical drive assembly 40 connectable to the proximal end of shaft 19 replacing portion 15 of housing 12.

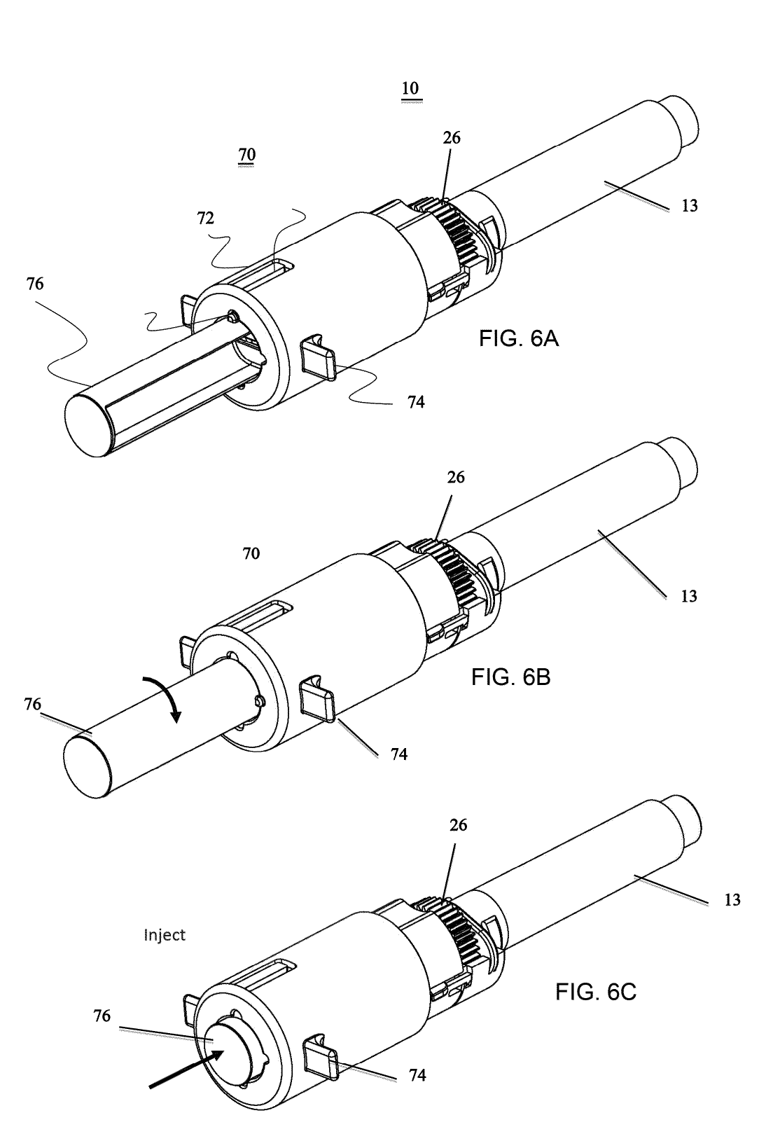

As is shown in FIG. 6a, a manual drive assembly 70 can include housing with finger holds 74 and a plunger pushrod 76. To mount assembly 70 on system 10, a user removes portion 15 of housing 12 (thereby unlocking shaft gear 26 from shaft 19) and mounts housing 72 in its place.

The configuration of assembly 70 shown in FIGS. 6a-c provides system 10 with syringe-like operability, i.e. the user can push or pull pushrod 76 to dispense or draw liquid into chamber 16. Pushrod 76 can include a rotatable locking mechanism which can be rotated between locked (FIG. 6a) and unlocked (FIG. 6b) positions. When unlocked, pushrod 76 can be depressed to deliver a medication (FIG. 6c) and if required withdrawn to draw liquid into chamber 16.

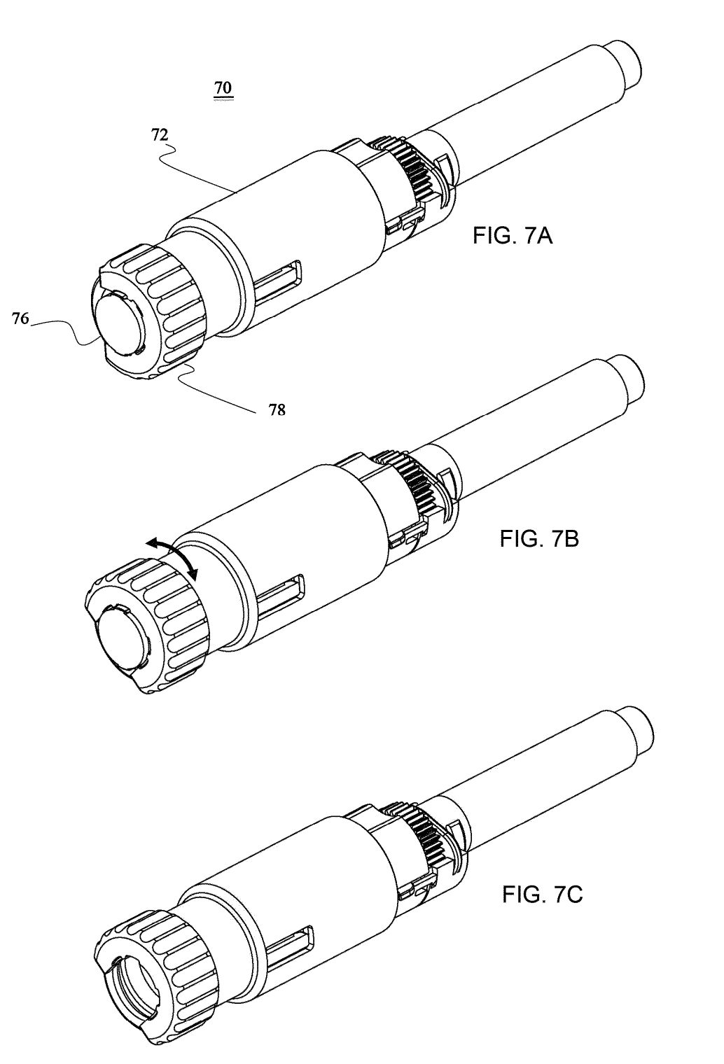

FIGS. 7a-c illustrates one embodiment of a spring-loaded drive assembly 70 which includes housing 72 and a spring loaded, push-button activated, plunger driving rod 76. This configuration of a spring-loaded drive assembly 70 is designed for delivery of a preset liquid volume, preferably as a single dose. The user unlocks the mechanism by rotating lock 78 (FIG. 7b), and depresses a button to deliver the medication (FIG. 7c).

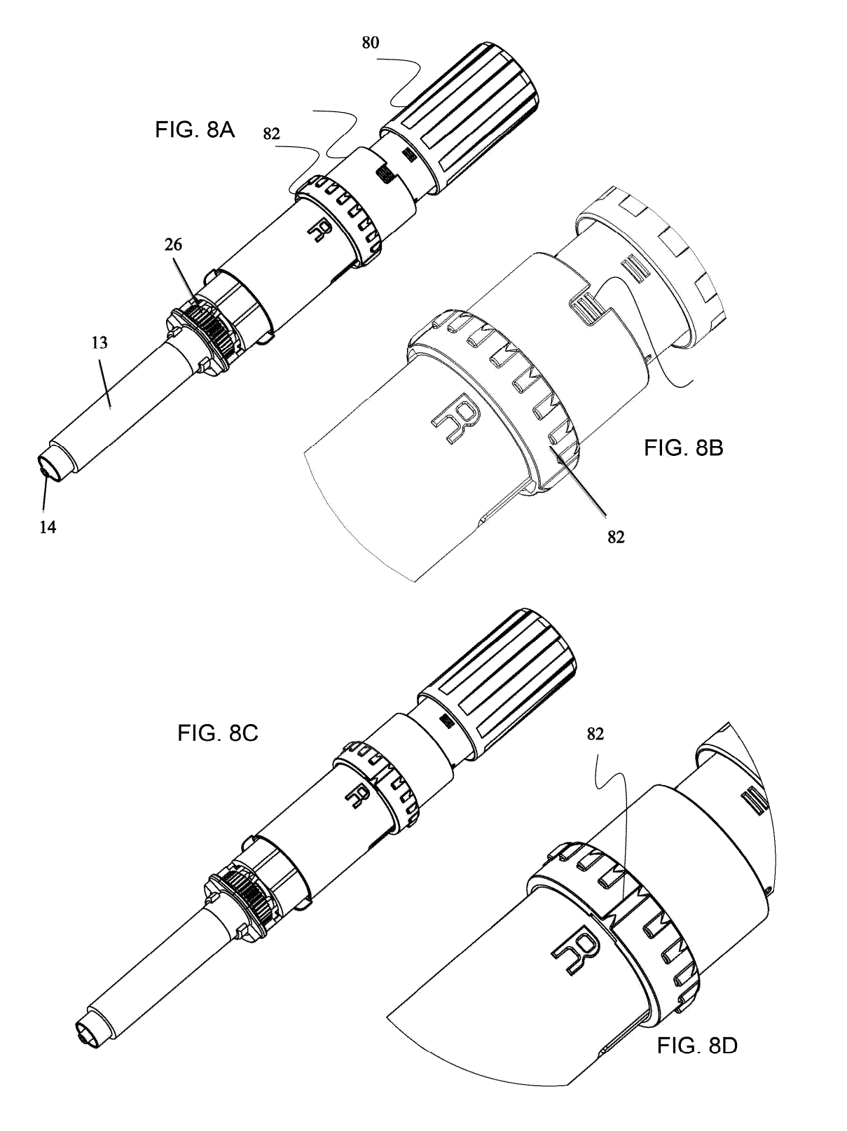

A multi-volume spring-loaded drive assembly 70 is illustrated in FIGS. 8a-d. This configuration uses a spring loaded, button activated mechanism, to deliver a volume of liquid selected by the user via knob 80. Knob 80 can be rotated to change the load on the spring thus changing the administration rate of liquid delivered by system 10. Housing 72 includes markings to indicate the rate of delivery and a lock/unlock status of the system (which is toggled via ring 82).

System 10 of the present invention can be used to prepare and deliver any liquid medicament. System 10 can be used as a bench top system, optionally placed within a hood or as a hand held unit in the treatment room setting.

System 10 is operated as follows: control unit 50 receives prescription information from the pharmacy database system via wired or wireless transmission. The prescription can include the following information: Prescriber, patient ID, recipe, mode and time of administration. The information is displayed to the user via display 56 graphically or textually, the user can flip forwards to the next prescription to prepare all the components in advance. The unit or pharmacy database system can also send the user text/graphics video alarms.

The user then removes a syringe assembly (portion 15 of housing with included plunger 18 and shaft gear 26, FIG. 2) form a sterile pack and connects it to drive mechanism 30 (with attached portion 15) and control unit 50 (FIG. 4a). A vial including a diluent is connected to port 14 via a vial adaptor such as 62. The vial can be scanned by a scanner built into control unit 50 to verify its contents. Control unit 50 is then activated via keypad 54 and motor 32 is activated to actuate plunger 18 to position zero (fully pushed in) in order to self calibrate system 10. A small volume of diluent is withdrawn from the vial and is then delivered back into the vial in order to fill dead volume within port 14. A preset volume of diluent is then withdrawn, and the diluent vial is disconnected by the user upon commend from control unit 50 (beep and/or display message). A vial containing a powder form of a drug (e.g. chemotherapy agent) is scanned by a scanner built into control unit 50 to verify its contents and is then connected by the user to the vial adaptor. System 10 then delivers the diluent in chamber 16 into the drug vial (while positioned upright, as verified by gravity switch or gyroscope of control unit 50). System 10 is then gently swirled to reconstitute the drug until an audible beep or message appears on display 56 (degree of swirling can be verified by an accelerometer built into control unit 50). System 10 is then placed upright and the reconstituted drug is withdrawn into chamber 16 (optionally, the reconstituted drug is pushed back into vial and withdrawn several times).

Portion 15 of housing is then disconnected from system 10 (as instructed by control unit 50) unlocking shaft 19 of plunger 18. A plunger driver (e.g. driver 70 of FIGS. 6-8) is then connected to proximal end of shaft 19 and to portion 13 of housing to enable manual or spring-driven delivery of the drug. Port 14 is plugged (directly or through a suitable connector) until use in cases where the drug is delivered to I.V. ports (bolus) or I.V. bags. For direct injection, port 14 is connected to a needle (via Luer lock).

Thus, the present invention provides a semi-automatic handheld system designed for easier, faster and safer preparation and administration of injectable vial drugs in or outside the pharmacy setting. The present system provides pharmacists and nurses with a safe, accurate, documented and easy to use compounding and administration system which can be used to administer reconstituted and/or compounded medication through an i.v. bag, an i.v. bolus, or via direct injection.

The present system can also wirelessly communicate with a hospital's Computerized Physician Order Entry (CPOE) systems for online computerized verification and documentation of each drug preparation. In home care setting, the system can connect via the internet to the patient care giver for relevant information.

As used herein the term “about” refers to ±10%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A system for medicament preparation and delivery comprising:
(a) a housing including a chamber for containing a liquid; and
(b) a plunger movable within said chamber for drawing and dispensing liquid, said plunger and said housing being configured such that said plunger is movable via a drive mechanism capable of engaging a side of said plunger wherein a top of a shaft of said plunger is connectable to a manually operable plunger interface.

2. The system of claim 1, further comprising a toggle for switching between movement of said plunger via said drive mechanism or movement of said plunger via said force to said top of said shaft of said plunger.

3. The system of claim 2, wherein said shaft of said plunger is configured with at least one groove for engaging said drive mechanism.

4. The system of claim 3, wherein said at least one groove is a spiral groove.

5. The system of claim 3, wherein said at least one groove engages a drive element of said drive mechanism.

6. The system of claim 3, wherein said toggle is capable of engaging said at least one groove.

7. The system of claim 1, further comprising said drive mechanism.

8. The system of claim 7, wherein said drive mechanism is attachable to a side wall of said housing.

9. The system of claim 8, wherein said drive mechanism includes a motor having a drive gear.

10. The system of claim 9, wherein said drive gear is a pinion.

11. The system of claim 9, wherein said drive gear is a worm drive gear.

12. The system of claim 9, wherein said drive gear is capable of engaging at least one groove in said shaft of said plunger.

13. The system of claim 9, wherein said shaft releasably engages a shaft gear which is capable of engaging said drive gear.

14. (canceled)

15. The system of claim 7, wherein said drive mechanism includes a control unit having a user interface for inputting parameters related to drawing and optionally dispensing of said liquid.

16. The system of claim 15, wherein said control unit includes an optical reader for scanning a drug vial.

17. The system of claim 15, wherein said control unit includes wireless communication capabilities or RFID.

18. The system of claim 1, wherein a proximal end of said shaft is configured capable of connecting to a spring driven mechanism.

19. The system of claim 1, further comprising spring driven mechanism connectable to a proximal end of said shaft of said plunger and said housing, said spring driven mechanism is capable of applying said force to said top of said shaft of said plunger.

20. The system of claim 19, wherein said spring driven mechanism drives said shaft for delivering a liquid from said chamber.

21. The system of claim 20, wherein a tension of a spring of said spring driven mechanism is user adjustable.

22. The system of claim 21, wherein connection of said spring driven mechanism to said shaft and said housing prevents said drive mechanism from moving said plunger.

23. A system for medicament preparation and delivery comprising:
(a) a housing including a chamber for containing a liquid; and
(b) a plunger movable within said chamber for drawing and dispensing liquid, said plunger including a spiral groove for engaging a drive gear of a drive mechanism mounted against a side of said plunger is connected at the to of the plunger, said drive gear being rotatable via said drive mechanism to rotatably slide said plunger within said chamber to increase or decrease a volume thereof; and
(c) a toggle for disengaging said drive gear from said spiral groove thereby enabling said plunger to slide within said chamber without rotating.

24. The system of claim 23, wherein said housing and said plunger are configured for enabling a top of said shaft of said plunger to interface with a manually operable plunger driver.
